SEQUENCE LISTING (9 sequences)

SEQ ID NO: 1 - DNA, 1533 bp, Bos taurus
  CDS (1)..(1533): DNA sequence coding for a calf intestinal alkaline phosphatase (ciAP)
atgaataagaaggtactgaccctttctgccgtgatggcaagtctgttattcggggcccacgcgcacgcggcgatcccagctgaggaggaaaaccccgccttctggaaccgccaggcagcccaggcccttgatgtagccaagaagttgcagccgatccagacagctgccaagaatgtcatcctcttcttgggggatgggatgggggtgcctacggtgacagccactcggatcctaaaggggcagatgaatggcaaactgggacctgagacacccctggccatggaccagttcccatacgtggctctgtccaagacatacaacgtggacagacaggtgccagacagcgcaggcactgccactgcctacctgtgtggggtcaagggcaactacagaaccatcggtgtaagtgcagccgcccgctacaatcagtgcaacacgacacgtgggaatgaggtcacgtctgtgatcaaccgggccaagaaagcagggaaggccgtgggagtggtgaccaccaccagggtgcagcatgcctccccagccggggcctacgcgcacacggtgaaccgaaactggtactcagacgccgacctgcctgctgatgcacagaagaatggctgccaggacatcgccgcacagctggtctacaacatggatattgacgtgatcctgggtggaggccgaatgtacatgtttcctgaggggaccccagaccctgaatacccagatgatgccagtgtgaatggagtccggaaggacaagcagaacctggtgcaggaatggcaggccaagcaccagggagcccagtatgtgtggaaccgcactgcgctccttcaggcggccgatgactccagtgtaacacacctcatgggcctctttgagccggcagacatgaagtataatgttcagcaagaccacaccaaggacccgaccctggcggagatgacggaggcggccctgcaagtgctgagcaggaacccccggggcttctacctcttcgtggagggaggccgcattgaccacggtcaccatgacggcaaagcttatatggcactgactgaggcgatcatgtttgacaatgccatcgccaaggctaacgagctcactagcgaactggacacgctgatccttgtcactgcagaccactcccatgtcttctcctttggtggctacacactgcgtgggacctccattttcggtctggcccccggcaaggccttagacagcaagtcctacacctccatcctctatggcaatggcccaggctatgcgcttggcgggggctcgaggcccgatgttaatggcagcacaagcgaggaaccctcgtaccggcagcaggcggccgtgcccctggctagcgagacccacgggggcgaagacgtggcggtgttcgcgcgaggcccgcaggcgcacctggtgcacggcgtgcaggaggagaccttcgtggcgcacatcatggcctttgcgggctgcgtggagccctacaccgactgcaatctgccagccccctccaccgccaccagcatccccgactga

SEQ ID NO: 2 - protein, 510 aa, Bos taurus
MNKKVLTLSAVMASLLFGAHAHAAIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPSTATSIPD

SEQ ID NO: 3 - DNA, 1527 bp, Homo sapiens
  CDS (1)..(1527): DNA sequence coding for a human placental alkaline phosphatase (hpAP)
atgaataagaaggtactgaccctttctgccgtgatggcaagtctgttattcggcgcgcacgcgcacgcggcgatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgactgataa

SEQ ID NO: 4 - protein, 507 aa, Homo sapiens
MNKKVLTLSAVMASLLFGAHAHAAIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTD

SEQ ID NO: 5 - DNA, 21 bp, Bos taurus
  misc_feature: Partial sequence from calf intestinal alkaline phosphatase (ciAP)
cttcggcgttcagtaacacgc

SEQ ID NO: 6 - DNA, 24 bp, Bos taurus
  misc_feature: Partial sequence from calf intestinal alkaline phosphatase (ciAP)
cctctagattatcagtcggggatg

SEQ ID NO: 7 - DNA, 21 bp, Bos taurus
  misc_feature: Partial sequence from calf intestinal alkaline phosphatase (ciAP)
ggtcacgtctgtgatcaaccg

SEQ ID NO: 8 - DNA, 21 bp, Bos taurus
  misc_feature: Partial sequence from calf intestinal alkaline phosphatase (ciAP)
cggttgatcacagacgtgacc

SEQ ID NO: 9 - DNA, 22 bp, Bos taurus
  misc_feature: Partial sequence from calf intestinal alkaline phosphatase (ciAP)
gctttcgaggtgaatttcgacc

(19) United States
(12) Patent Application Publication
(10) Pub. No.: US 2005/0153338 A1
(43) Pub. Date: Jul. 14, 2005

(54) Method for obtaining circular mutated or chimeric polynucleotides

(76) Inventors: Peter Kratzsch, Antdorf (DE); Von der Eltz Herbert, Weilheim (DE); Rainer Schmuck, Benediktbeuern (DE); Boenitz-Dulat Mara, Muenchen (DE)

Correspondence Address: Roche Diagnostics Corporation, 9115 Hague Road, PO Box 50457, Indianapolis, IN, 46250-0457, US

(21) Appl. No.: 11/004,679

(22) Filed: Dec. 3, 2004

(30) Foreign Application Priority Data
Dec. 4, 2003 (EP) EP 03027838.6

(51) Int. Cl. C12Q 1/68; C12P 19/34

(52) U.S. Cl. 435/6; 435/91.2

(57) ABSTRACT

A quick, simple, and efficient in vitro method for generating a random chimeric or mutated gene or genes in a circular polynucleotide molecule. The method is based on hybridization of random DNA fragments to a closed, circular DNA template, elongation of the fragments by a DNA polymerase, and ligation of the fragments to each other by a DNA ligase resulting in a recombined, closed circular DNA molecule which functions as a new template in a next cycle of chimerization. The degree of recombination can be controlled by the number of cycle repetitions, the amount of different templates and fragments, and the ratio of the latter. This procedure is referred to as polymerase chain chimerization (PCC). The PCC method is useful for generating new, improved bio-molecules.

### FIELD OF THE INVENTION

The present invention relates to a quick, simple and efficient in vitro method for generating mutated circular polynucleotide molecules. The method is based on providing a closed circular DNA polynucleotide template comprising a first gene of interest, providing fragments of a double-stranded DNA target polynucleotide comprising a second gene of interest, capable of hybridizing to the first gene of interest, wherein the fragments have free 3′-OH ends and phosphorylated 5′-ends, generating single strands of both said template polynucleotide and said fragments of the target polynucleotide, annealing the fragments of the DNA target polynucleotide to the circular polynucleotide, elongating the fragments, e.g., by using a DNA polymerase, ligating the elongated fragments to each other, e.g., by using a DNA ligase. These steps result in a recombined or mutated circular daughter strand DNA. To further increase the frequency of mutation or recombination the steps of annealing, elongation and ligation are repeated 1 to 100 times. A multitude of mutated or chimeric polynucleotides is easily obtained by this method. The invention also discloses host cells transformed with a mutated polynucleotide obtained by the above method and it discloses methods for obtaining recombinant biomolecules based on such mutated polynucleotides.

### BACKGROUND

There is a major interest in bio-molecules for medical, industrial and scientific applications (Powell, K. A., et al., Directed Evolution and Biocatalysis, Angew. Chem. Int. Ed. Engl. 40 (2001) 3948-3959) (Kirk, O., et al., Industrial enzyme applications, Curr. Opin. Biotechnol. 13 (2002) 345-351).

Some applications require bio-molecules with very special properties or features which do not exist or are very hard to find in nature. One way to generate such bio-molecules mimics the principle of natural evolution. Random mutants of a bio-molecule are generated and a mutant with the desired property is selected by an appropriate screening process. This kind of approach is termed “directed evolution” (Arnold, F. H., and Volkov, A. A., Directed evolution of biocatalysts, Current Opinion in Chemical Biology 3 (1999) 54-59).

Various techniques can be used to create these mutants (Vasserot, A. P., et al., Optimization of protein therapeutics by directed evolution, Drug Discov Today 8 (2003) 118-126) (Graddis, T. J., et al., Designing proteins that work using recombinant technologies, Curr. Pharm. Biotechnol. 3 (2002) 285-297) (Brakmann, S., Discovery of superior enzymes by directed molecular evolution, Chembiochem. 2 (2001) 865-871).

The methods used today to generate bio-molecules with desired properties can be roughly divided into techniques introducing point mutations and techniques in which similar but not identical polynucleotide sequences are recombined.

The first type of methods leads to mutated polynucleotides by introducing random point mutations into the sequence of interest, e.g., based on error prone polymerase chain reaction (PCR) (Cadwell, R. C., and Joyce, G. F., PCR Methods Appl. 2 (1992) 28-33), use of so-called mutator strains (Greener, A., et al., Methods Mol. Biol. 57 (1996) 375-385), or on treating the DNA with chemical mutagens (Deshler, J. O., Gen. Anal. Techn. Appl. 9 (1992) 103-106).

The second type of technique which leads to chimeric polynucleotides comprises the recombination of genes or gene-fragments derived from different variants of a gene.

In WO 01/34835 techniques are described for the recombination of functional domains.

The most commonly used recombination method for optimization of bio-molecules is called “DNA Shuffling” (U.S. Pat. No. 5,834,252; U.S. Pat. No. 5,605,793; U.S. Pat. No. 5,830,721; U.S. Pat. No. 5,837,458; U.S. Pat. No. 5,811,238). This method comprises the creation of double-stranded fragments from different double-stranded DNA templates wherein said different DNA templates contain homologous and heterologous areas. The reassembly of the random fragments is performed by a PCR-like reaction without further addition of primers. The occurrence of overlapping domains of the DNA sequences which can function as primers/templates to each other is essential for a successful reassembly. Due to the poor yield of the recombined DNA sequence after reassembly, it usually has to be amplified by an additional PCR reaction and afterwards it has to be cloned in an appropriate expression system. The gene products can then be transformed into appropriate host cells, expressed and then screened for an enhanced or new property.

Methodological alternatives to DNA shuffling are random priming recombination (RPR) (Shao, Z., et al., Nucleic Acids Res. 26 (1998) 681-683) and the staggered extension process (StEP)( Zhao, H., et al., Nat. Biotechnol. (1998) 16258-16261).

Site directed mutations can be achieved by the methods disclosed in WO 02/16606; WO 99/35281. These methods use circular DNA as a template and site directed mutations are introduced by use of specially designed primers already comprising point mutations.

WO 01/29211 also describes a recombination method. The template polynucleotide according to this method is not integrated into a circular DNA, making it necessary to insert the recombined DNA in a suitable expression system after recombination.

Another method for obtaining chimeric genes is described in WO 00/09697. The method disclosed there achieves a larger diversity of recombined DNA molecules by using various types of linear assembly DNA matrices.

Despite the significant progress in the various methods leading to mutant and especially to chimeric polynucleotides there is still a tremendous need to provide a method for generating chimeric polynucleotides that would help to at least partially avoid the known drawbacks of the state of the art procedures.

The aim of the present invention was therefore to develop a simple and efficient DNA recombination method for obtaining chimeric polynucleotides by reducing the number of working steps as much as much as possible while achieving a high level of diversity in the resulting chimeric polynucleotides.

It has been found that the disadvantages of the methods known in the art can be at least partially overcome by the methods, host cells and recombinant bio-molecules according to the present invention.

### SUMMARY OF THE INVENTION

The present invention relates to a method for generating a mutated or chimeric polynucleotide, the method comprising providing a closed circular DNA polynucleotide template comprising a first gene of interest, providing fragments of a double stranded DNA target polynucleotide comprising a second gene of interest, capable of hybridizing to the first gene of interest, said fragments having free 3′-OH ends and phosphorylated 5′ ends, generating single strands of both said template polynucleotide and said fragments of the target polynucleotide, annealing said fragments of the target polynucleotide to said template polynucleotide, elongating the annealed fragments of the target polynucleotide by a DNA polymerase, ligating the nicks between the elongated fragments by using a DNA ligase thereby generating a chimeric full length circular DNA molecule, and then using the said chimeric full length circular DNA molecule repeatedly as a closed circular DNA template polynucleotide by again generating single strands, annealing, elongating and ligating as described before. Since this technology in some aspects resembles the very popular polymerase chain reaction the term polymerase chain chimerization (PCC) is proposed.

The present invention also relates to a method for obtaining a transformed host cell by introducing a mutated or chimeric polynucleotide obtained according to a method of the present invention.

The present invention also refers to a host cell containing a mutated or chimeric polynucleotide produced by a method according to the present invention.

A further embodiment concerns a method for obtaining a recombinant bio-molecule which comprises obtaining a mutated or chimeric polynucleotide by use of a method according to this invention, transforming said mutated or chimeric polynucleotide into an appropriate host, expressing the recombined gene, and screening for said recombinant bio-molecule.

### BRIEF DESCRIPTION OF THE DRAWINGS

FIG. 1. Schematic of a method according to the present invention

In FIG. 1 the inventive method using a double-stranded closed circular polynucleotide comprising a first gene of interest (striped arrow) and fragments derived from a double-stranded target DNA (bold arrow) is shown.

The polynucleotides are provided and mixed with all reagents required, then the steps of melting, annealing, polymerization and ligation are performed.

FIG. 2. Amino acid sequences for hAP and ciAP

The amino acid sequence for calf intestinal alkaline phosphatase is shown in the first row and the corresponding sequence of human placental alkaline phosphatase is shown in the second row.

FIG. 3. Fragmentation of target DNA

The results of target DNA fragmentation with DNAse I according to Example I are shown in FIG. 3.

FIG. 4. Nucleic acids obtained after 20 cycles of chimerization

5 μl of sample, obtained as described in Example 1, were applied to a 1% agarose gel. After electrophoresis the DNA bands were stained with ethidium bromide. Stained bands are shown.

FIG. 5. SEQ ID NO: 1

FIG. 6. SEQ ID NO: 3

FIG. 7. SEQ ID NO's: 5-9

### DESCRIPTION OF THE INVENTION

In a first preferred embodiment the present invention relates to a method for generating a mutated or chimeric polynucleotide, the method comprising providing a closed circular polynucleotide template comprising a first gene of interest, providing fragments of a double stranded DNA target polynucleotide derived from a second gene of interest capable of hybridizing to the first gene of interest said fragments having free 3′-OH ends and phosphorylated 5′ ends, generating single strands of both said template polynucleotide and said fragments of the target polynucleotide, annealing said fragments of the target polynucleotide to said template polynucleotide, elongating the annealed fragments of the target polynucleotide by a DNA polymerase, ligating the nicks between the elongated fragments by using a DNA ligase thereby generating a chimeric full length circular DNA molecule. The mutated or chimeric full length circular DNA molecule may then be used repeatedly as a closed circular polynucleotide template in the next cycles of chimerization by again generating single strands, annealing, elongating and ligating as described before. For convenience this method is referred to as a chimerization method, although other advantageous mutants without a recombination event are also obtained by the inventive method.

The term “mutated or chimeric gene” is used to indicate that mutated or chimeric polynucleotides are obtained at a high frequency by using the method according to the present invention. As known in the art mutations are for example point mutations, deletions and insertions. A chimeric polynucleotide in the sense of the present invention is any gene of interest comprising at least 15 consecutive polynucleotides derived from two different genes of interest. Thus although the major advantage of the present invention is the high frequency of chimerization which is obtained, it also has the additional and welcome side effect that mutated polynucleotides are also frequently generated when using the chimerization method of the present invention. The term “or” is used to indicate that polynucleotides are obtained comprising mutations alone, mutations as well as chimeric sequences, or chimeric sequences in the absence of further mutations.

Preferably the gene selected by a method according to the present invention is a chimeric gene with or without additional mutations.

A “gene of interest” relates to any polynucleotide sequence exhibiting a property which can be selected or screened by any appropriate screening or selection procedure. The present invention also makes use of at least a “first” and a “second” gene of interest.

The term “a” in relation to the first or to the second gene of interest must not be understood as exclusively referring to a single gene. Rather the present invention can also be successfully applied using several different template as well as several different target genes. Preferably all these template and target genes represent variants of a single gene. It is, however, preferred to use three, two or only one template or target gene or genes, respectively.

Obviously the molar ratio of the template gene to fragments of a target gene will influence the degree of chimerization achieved. Molar ratios (fragments/template) from 15-1000 are recommended, with ratios of 10-500, 20-400 and 30 to 300 being more preferred.

The first and the second gene of interest must be at least partially homologous in order to allow hybridization and annealing as required for the inventive method. The skilled artisan will have no problems in selecting appropriate pairs of genes for performing the method of this invention.

In order to obtain an adequate frequency of hybridization or annealing the template and the target gene must have a homology of at least 40%. Preferably the homology between the two genes is at least 50%. The homology is preferably higher and at least 60% or at least 70%. Most preferably, the homology is 80% or more, as determined in the method described below.

The homology between the first and the second gene of interest is preferably analyzed by the PileUp program of the GCG Package, Version 10.2 (Genetics Computer Group, Inc.). PileUp creates a multiple sequence alignment using a simplification of the progressive alignment method of Feng, D. F., and Doolittle, R. F., J. Mol. Evol. 25 (1987) 351-360, and the scoring matrixes for identical, similar, or different amino acid residues are defined accordingly. This process begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments that include increasingly dissimilar sequences and clusters, until all sequences have been included in the final pairwise alignment.

For convenience sequence identity may also be assessed when comparing the template and the target gene. In such comparisons individual nucleotides at corresponding sequence positions are compared with one another. The sequence identity is preferably at least 40% for at least one partial sequence of 30 nucleotides in length. More preferably the sequence identity is higher and at least 50% or even more preferably at least 60%.

Many related genes occurring in nature serve the same purpose in different individuals as well as in different species. By way of example, hemoglobin is used to transport oxygen in blood. Within the human species hemoglobin is known to be present with slightly different sequences (known as alleles), whereas hemoglobin in other mammals exhibits more differences on a sequence level. These genes thus represent naturally occurring variants of a single gene or gene locus. Preferably both a template and a target gene represent naturally occurring variants of a single gene.

As the skilled artisan will appreciate, any combination of template and target gene will be appropriate as long as fragments of the target gene hybridize to the template gene. The conditions of hybridization may be varied, e.g., by changing buffer conditions. According to the present invention annealing is preferably performed in a buffer consisting of 0.2M Tris-HCl, 0.02M MgSO₄, 0.1 M KCl, 0.1 M (NH₄)₂SO₄, 1% Triton X-100, serum albumin (BSA)/ml at pH 8.8. Any pair of template and target gene, which yields a sufficient number of annealing events in the first round of chimerization in said buffer, is appropriate for performing the chimerization method according to this invention.

The first gene of interest is provided as an integral part of a closed circular polynucleotide. As a matter of convenience the first gene of interest as comprised in a closed circular DNA polynucleotide is also referred to as a “template” gene.

Any type of circular polynucleotide (RNA or DNA) or even circular pseudopolynucleotides comprising nucleotide analogues like for example peptide nucleic acids (PNAs) may be used as a first polynucleotide. In an advantageous and preferred embodiment, the first polynucleotide comprising the template gene is a deoxyribonucleic acid (DNA). The closed circular polynucleotide is most preferably double-stranded DNA.

There are numerous methods for cloning a certain gene and inserting it in a suitable expression system which do not have to be recited here. For convenience it is mentioned that the skilled artisan is quite familiar with these methods which are summarized for example in Current Protocols in Molecular Biology Volume 1-4, Edited by Ausubel, F. M., et al., Massachusetts General Hospital and Harward Medical School by John Wiley & Sons, Inc.

The template gene can easily be amplified and at the same time methylated by transforming the plasmid into a suitable host strain, inoculating the plasmid-carrying strain for an appropriate time and isolating the plasmid from the cell culture. The use of a methylated circular polynucleotide provides an additional advantage. After the chimerization is completed in a method according to the present invention the methylated DNA (i.e. the starting material) can be easily removed by using specific restriction enzymes which cut out only methylated DNA. As the initial template is methylated DNA and only non-methylated circular mutated or chimeric DNA molecules are produced, the starting template can be eliminated by using an enzyme like DpnI that only cleaves methyl-DNA. The most preferred way to perform the inventive method thus comprises using a methylated closed circular DNA as the polynucleotide containing the first gene of interest.

Preferably the polynucleotide sequence of the first gene of interest is the full length polynucleotide for this gene. As the skilled artisan will appreciate, this gene may however, be shortened to represent only partial sequences of a gene. The minimum length required is a polynucleotide which exhibits a biological activity or function which can be identified by any appropriate screening or selection method.

The second gene of interest is also referred to as a “target” gene for convenience. The target gene must be available in a double-stranded form and preferably as DNA double-strands. Exponential amplification during the various rounds of chimerization is only achieved if the fragments derived from the target gene comprise fragments of both the sense as well as of the anti-sense strand.

The target gene or polynucleotide is not provided as a full length continuous molecule but rather in a fragmented form. As the skilled artisan will appreciate, appropriate fragments of a double-stranded DNA polynucleotide may be generated by various means. The methods for providing fragments of a double-stranded target polynucleotide are not critical as long as care is taken that at least some of the fragments thus generated are capable of hybridizing to the first gene of interest with the additional requirement that the fragments provided must have a 3′OH and a phosphate residue at the 5′-end, respectively. Most preferred are methods leading to a random fragmentation of the target gene.

Random fragments can be obtained by numerous methods known to the skilled artisan. One possibility would be to amplify the DNA sequence of interest by PCR and randomly cut the PCR product by an endonuclease resulting in fragments with the required 3′-OH and 5′-phosphate ends. For example, digestion can be accomplished in a very short time with DNAse I and the degree of digestion can be monitored on an agarose gel. After the desired degree of fragmentation is achieved the reaction can be stopped, e.g., by heating the sample to 95° C. for 5 minutes thus destroying the nuclease. The DNAse I can also be separated from the fragments by various methods i.e. using polymerase chain reaction (PCR) purification kits which are commercially available. Another possibility would be to use very short primers for random annealing in a PCR reaction with the second DNA sequence of interest as the template resulting in PCR products of different lengths and the 5′ ends of the fragments thus obtained can then be phosphorylated with an appropriate kinase. Usually fragments with a length between 8 and 1000 base pairs (bp) should be used. In a preferred embodiment the majority (more than 50%) of the fragments used will be between 10 and 300 bp.

The terms annealing, elongating and ligating as used in the present invention have the meanings the skilled artisan attaches thereto.

The method according to the present invention may be repeated 1-100 times. Whereas in the first round of chimerization only the closed circular polynucleotide template comprising the first gene of interest is present, an ever increasing number of already mutated or chimeric closed circular DNA polynucleotides will be present in each of the following cycles which will again act as a template for the next cycle according to the present invention. The use of polynucleotides that are already mutated or chimeric polynucleotides as templates in the consecutive rounds of chimerization further increases the total number of different mutated or chimeric molecules.

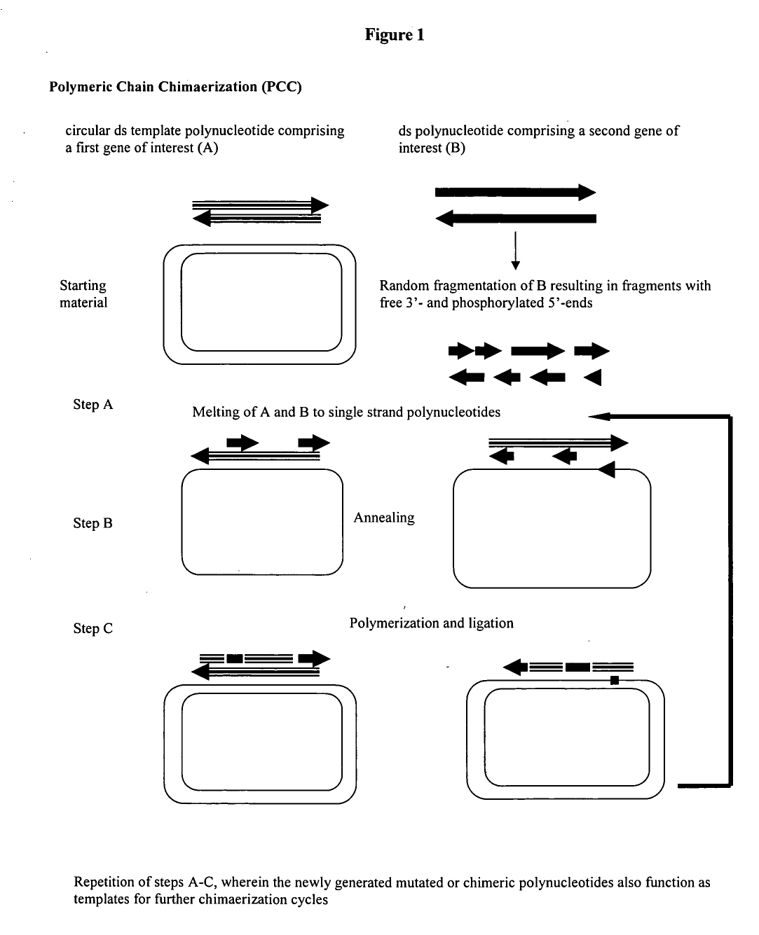

The method according to the present invention is further illustrated in FIG. 1. In a first step single-chain molecules are generated for both the template as well as the target polynucleotide. In a second step single-stranded DNA fragments of a target gene or sequence of interest are annealed to a template polynucleotide which is incorporated in a circular closed single strand polynucleotide molecule. The annealed fragments are elongated by a DNA polymerase until the enzyme meets an already bound fragment. The DNA polymerase then drops of the DNA strand leaving a nick between the elongated 3′-OH ends of a fragment and the 5′-phosphate ends of a fragment. The gap or nick is closed by a DNA ligase which ligates the 3′-OH extension to the phosphorylated 5′ end of the next fragment bound. A double-stranded DNA comprising two closed circular DNA molecules is obtained as the final result of such a cycle of chimerization. It is obvious to the skilled artisan that the number of annealed fragments determines the degree of chimerization of the DNA sequences of interest. The method according to the present invention provides the major advantage that the double-stranded closed circular polynucleotide obtained in the first or more generally in a previous round of chimerization can be directly used in a next round of chimerization. In order to perform a next round of chimerization the double strand obtained in the first round is melted and both single stranded circular molecules now function as a template polynucleotide in the next cycle of chimerization. Preferably two to fifty cycles of chimerization as described above are performed. This leads to an ever increasing number of recombinations resulting in a great diversity of mutated or chimeric polynucleotides. Between 15 and 35 cycles of chimerization are most preferably used.

The efficient and convenient method of chimerization according to the present invention can easily be performed in a single mixture of reagents which contains all required reactants. Details of preferred reactants are given further below and in the example section. If required the inventive method can also be performed in steps, e.g. fragments of the target polynucleotide could be added at the beginning and again after an appropriate number of chimerization cycles. Such and similar modifications are of course possible within the scope of the present invention.

The chimerization can be achieved by carrying out a PCR-like reaction using a closed circular polynucleotide comprising the template gene and fragments of the target gene with both orientations as primers. Preferably the reaction is carried out at elevated temperatures. The use of a thermo-stable DNA polymerase and of a thermo-stable DNA ligase, like DNA polymerases and ligases obtained from organisms like Pyrococcus furiosus, Thermos aquaticus, etc. is also preferred. It is also advisable and preferable to use a DNA polymerase with proofreading activity to ensure that unwanted polymerization errors beyond the DNA sequence of interest are avoided. This is also advantageous for retaining an intact expression system.

As described above the product of a chimerization cycle functions as a template for the remaining fragments in the reaction mixture and the chimerization cycle can be repeated. This means that as in a normal PCR reaction an exponential amplification of the mutated or chimeric DNA sequences is achieved and furthermore that the number of polynucleotides is exponentially amplified. The ratio of starting template to newly generated DNA increases in favor of the chimeric molecule in each cycle. This also substantially reduces the risk or likelihood of obtaining parental clones after transformation of the mutated or chimeric polynucleotides into an appropriate host cell.

A standard PCR-like chimerization cycle according to the present invention for example utilizes the following reagents: a buffer system in which the chosen polymerase and ligase work, dNTP's, polymerase, ligase, ATP (or another cofactor for ligase), template and fragments of the target polynucleotide; and comprises the following steps: incubation at 72° C. to ligate undesired nicks in the circular template, 30 seconds at 95° C. to m double-stranded DNA into single-stranded DNA, 1 minute at 50° C. to anneal the fragments to the template, 8 minutes at 72° C. to elongate the fragments by DNA polymerase, 30 seconds at 95° C. to remove bound DNA polymerase from the template and 8 minutes at 72° C. to ligate the fragments to each other and to eliminate any nicks. The chimerization cycle is preferably repeated about 20 to 25 times. Finally the reaction mixture is cooled to 4° C.

As the described method is based on the use of closed circular polynucleotides and results predominantly in closed circular chimeric DNA molecules, it is evident that it is advantageous to use plasmids as closed circular DNA template molecules. Such a plasmid preferably comprises a suitable expression system for the DNA sequence or gene of interest. The template polynucleotide is preferably a double-stranded closed circular plasmid comprising an origin of replication, a promoter for gene expression, a gene for selection and a first gene of interest. As the skilled artisan will readily appreciate preferably only that part of the polynucleotide sequence which comprises the gene of interest is recombined and the rest of the DNA sequence, i.e. the expression system or plasmid part of the sequence is left untouched. Two or more different plasmids containing the DNA sequence of interest may also be used in a method according to this invention.

The working steps usually needed by the other methods known from the art, e.g. requiring the insertion of the newly mutated or chimeric DNA molecules into an appropriate expression system are not necessary in the case of the mutated or chimeric DNA polynucleotides produced by a method according to this invention are already incorporated in an expression system. Hence additional steps such as the preparation of vectors and insertion using suitable restriction enzymes and ligation of the later are avoided. This makes the inventive method much more convenient and simple to use.

The result of the chimerization PCR i.e. PCC can be monitored by agarose gel electrophoresis. A polynucleotide of the desired size (identical to the template) should be visible on the agarose gel.

Preferably the present invention also relates to a method for obtaining a transformed host cell by introducing a mutated or chimeric polynucleotide obtained according to a method of the present invention into said host. Bacteria like E.coli or Bacillus strains are preferred as host strains. Such transformations have been found to be quite successful and produce a high yield of transformants.

The mutated or chimeric circular DNA obtained in a method according to the present invention can be subsequently transformed into an appropriate host cell without any further working steps. As described above, this transformation can optionally incorporate a step for removing a methylated template polynucleotide. However, this step also does not require further purification. It is therefore preferable to directly use the mutated or chimeric polynucleotides obtained in a method according to this invention without prior purification to transform a host cell.

Of course a host cell obtained by transformation with a mutated or chimeric polynucleotide obtained by the method of the present invention represents a further preferred embodiment of the present invention.

The present invention also relates to a method for obtaining a recombinant bio-molecule, the method comprising: obtaining a mutated or chimeric polynucleotide by use of a method according to the present invention, transforming said mutated or chimeric polynucleotide into an appropriate host, expressing the recombined gene, and screening for said recombinant bio-molecule.

After isolation of individual clones, inoculation and expression of the mutated or chimeric gene products the new bio-molecules can be screened in order to identify clones containing a recombinant gene with the desired property.

Screening may be performed for any selectable property. Preferably the bio-molecule is an expression vector and expression vectors that lead to a higher protein yield are selected. The bio-molecule is most preferably a polypeptide and the selection process is for example simply based on a selectable property of such a polypeptide. Such a recombinant polypeptide is most preferably an enzyme. As the skilled artisan will appreciate, different enzymatic properties are quite easily selected for once the new (mutated or chimeric) polynucleotide has become available and has been expressed as described above.

As the skilled artisan will readily appreciate the methods disclosed herein are quite advantageous for various reasons, e.g. they obviate the necessity for an amplifying PCR reaction that is still required by state of the art methods especially in order to generate mutated or chimeric DNA molecules at a reasonable frequency and no purification step prior to transformation is necessary. It is also quite obvious that by omitting the amplification PCR no pre-selection of mutated or chimeric polynucleotide occurs. This results in a larger diversity of mutated or chimeric polynucleotides entering into transformation which also results in a larger diversity of genetic variants for screening and selection.

The following examples, references, sequence listing and figures are provided to aid the understanding of the present invention, the true scope of which is set forth in the appended claims. It is understood that modifications can be made in the procedures set forth without departing from the spirit of the invention.

### EXAMPLES

### Example 1

### Chimerization of Human Placental Alkaline Phosphatase (hpAP) with Calf Intestinal Alkaline Phosphatase (ciAP)

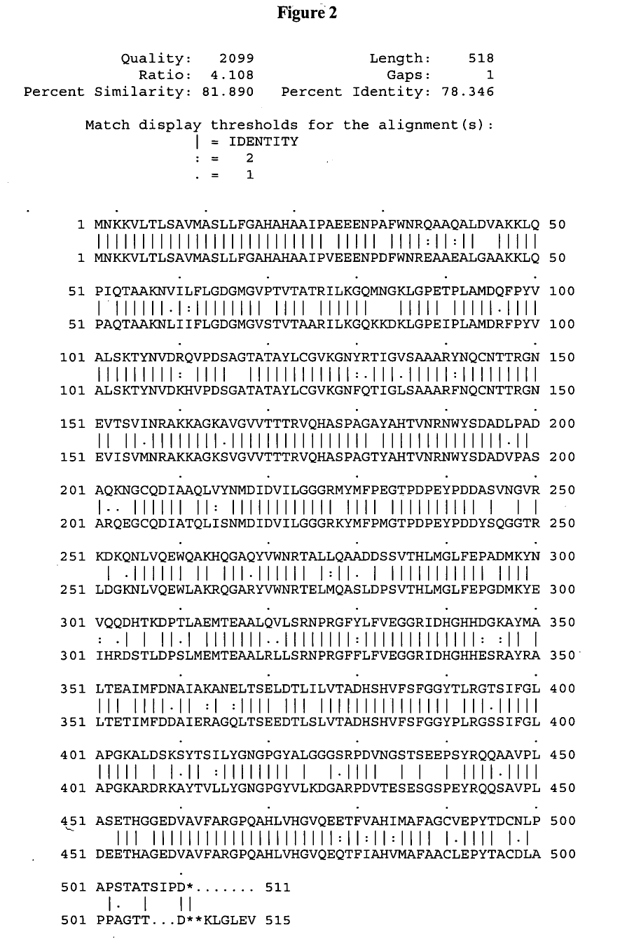

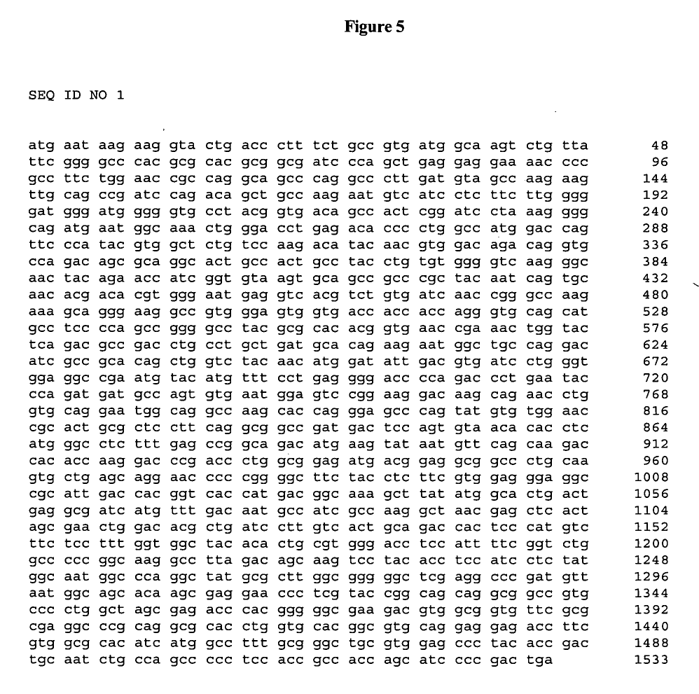

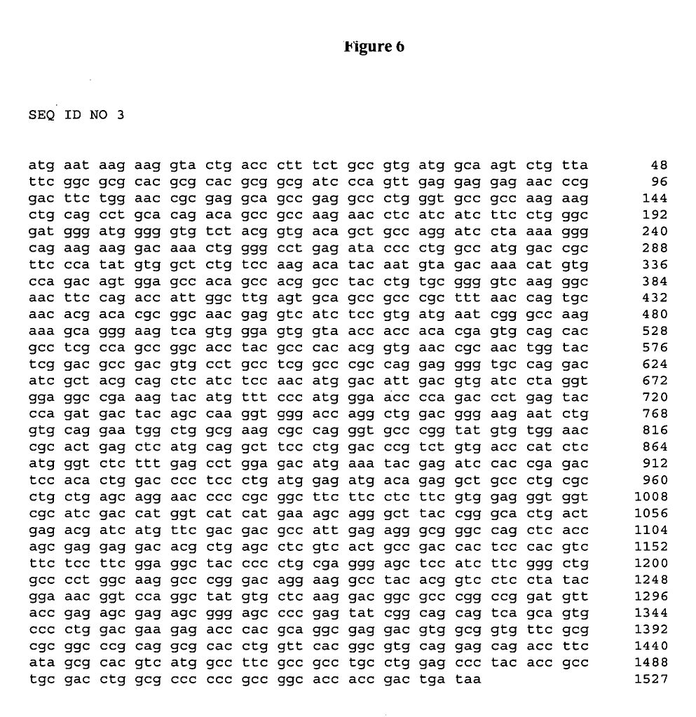

The genes of ciAP and hpAP exhibit a homology of about 80% to each other (see SEQ ID NO's 1 and 3, respectively, in FIGS. 5 and 6). Both the amino acid sequences are shown in FIG. 2 and in SEQ ID NO's 2 and 4, repectively. The distribution of homologous and heterologous areas is nearly random over the whole gene, making it easy to monitor the degree of chimerization of both the genes.

The two genes were cloned into the plasmid pkk 177 with an ampicillin resistance gene where they were under the control of a pmgl promoter (see patent application WO 88/09373) and transformed into the E.coli strain XL-blue-MRF'.

For the following experiment hpAP was used as a first gene of interest.

The random fragments of the target gene were generated from the ciAP gene.

Template Gene (Plasmid):

The E.coli strain carrying the plasmid with the hpAP gene was inoculated over night at 37° C. The plasmid was isolated using a Roche High Pure Plasmid Isolation Kit Id. 1754777 and the concentration determined by UV at 260 nm. A solution of 5 ng DNA/μl water was prepared.

Target Gene (Fragments):

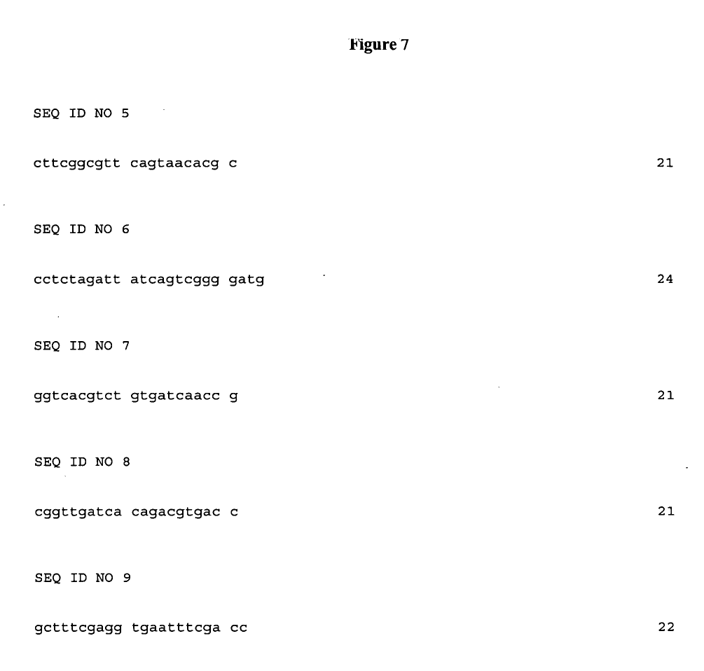

An aqueous solution containing the plasmid carrying the ciAP gene was prepared using the same method as for the template. The ciAP gene was amplified by PCR using suitable primers (see SEQ ID NO's 5 and 6, respectively, in FIG. 7).

The resulting PCR product was purified using a Roche PCR purification kit and the DNA concentration was determined.

Fragmentation:

The ciAP gene was subjected to DNAse I digestion. The following preparation was made:
30 μl ciAP (100 ng DNA/μl water)
1.5 μl DNAse I (0.1 U/μl; Roche Cat. No. 776785 diluted with 1× buffer Cat. No.1417991)
8 μl buffer (10× Puffer Cat. No.1417991)
40.5 μl water

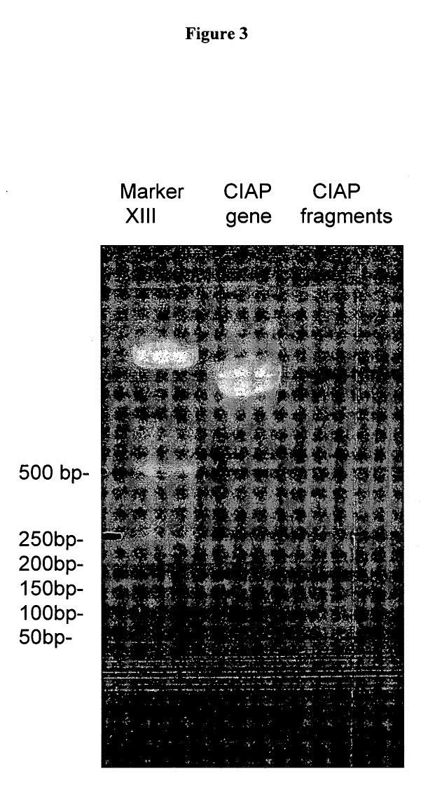

After 10 minutes incubation at room temperature the preparation was quickly frozen on dry ice to stop DNAse I digestion. 5 μl sample were analyzed on a 1% agarose gel to monitor digestion (see FIG. 3).The cloud of fragments proved that the majority of fragments was about 50 bp in size. DNAse I was inactivated by heating the remaining preparation at 95° C. for 10 minutes. The DNA was purified by the Roche PCR purification Kit and the DNA concentration was determined at 260 nm.

Chimerization-PCR (PCC):

| | neg. control | |
|---|---|---|
| | (without fragments) | PCC-sample |
| hpAP template 5 ng/μl | 2 μl | 2 μl |
| ciAP fragments 19 ng/μl | — | 10 μl |
| 2.5 U/μl Pfu polymerase | 1 μl | 1 μl |
| 10 × Pfu polymerase buffer | 5 μl | 5 μl |
| 4 U/μl Pfu ligase | 1 μl | 1 μl |
| 40 mM dNTP | 1 μl | 1 μl |
| 5 μM/μl ATP | 1 μl | 1 μl |
| water | 39 μl | 29 μl |

2.5 U/μl Pfu polymerase from Stratagene Cat. No. 600252
10 × Pfu polymerase buffer from Stratagene Cat. No. 600252
4 U/μl Pfu ligase from Stratagene Cat. No. 600191
40 mM dNTP = 10 mM dATP, 10 mM dCTP, 10 mM dTTP, 10 mM dGTP
5 μM/μl ATP from Roche Cat. No. 1 140965

Temperature Program:
a) 10 minutes 72° C.
b) 30 seconds 95° C.
c) 1 minute 55° C.
d) 8 minutes 72° C.
e) 30 seconds 95° C.
f) 8 minutes 72° C.
g) 20-fold repetition of steps b) to f)
h) 4° C.

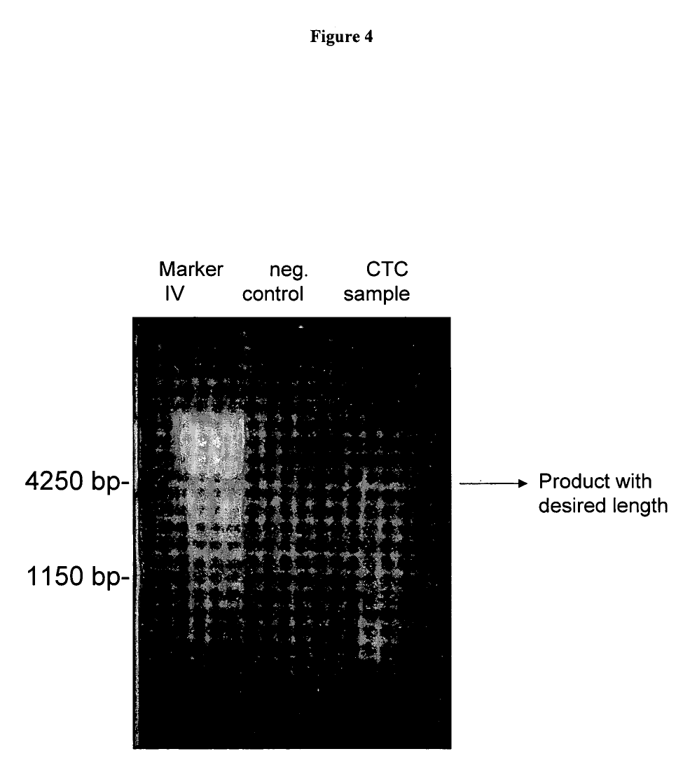

After the thermo-cycling 5 μl of the PCC-PCR products were applied to a 1% agarose gel (see FIG. 4).

Results: The PCC-sample shows a band having the desired size, whereas the neg. control shows none.

### Example 2

### Transformation

Transformation was done by electroporation of both samples PCC and neg. control respectively into the E.coli XL-MRF' strain (Stratagene Cat. No. 200158). Afterwards 100 μl of the PCC-preparation and 1 ml of the neg. control were plated out on an LB agar plate containing 100 μg/ml ampicillin. Cells were incubated over night at 37° C.

The results of this transformation experiment in terms of the number of growing clones are given in the following Table:

| | Neg. control-sample | PCC-sample |
|---|---|---|
| Clones | 0 | 640 |

20 clones where randomly picked from the PCC-sample plate and separately inoculated in LB-amp medium overnight at 37° C. The plasmids of the 20 samples were isolated using a Roche High Pure Plasmid Isolation Kit Id. 1754777 and sequenced using an ABI Prism Dye Terminator Sequencing Kit and ABI 3/73 and 3/77 sequencer (Amersham Pharmacia Biotech). The sequencing primers used were all based on the ciAP gene (see SEQ ID NO's 5, 7, 8, and 9, respectively, in FIG. 7).

### Example 3

### Testing Clones for AP Activity

The AP activity was determined as follows: 90 μl cell suspension of the overnight culture was mixed with 10 μl of B-PER (Bacterial Protein Extraction Reagent, Pierce Cat. No. 78248) to disrupt the cells. 50 μl of this mixture were added to 90 μl reagent (Roche Cat. No. 2173107). Active AP releases p-nitrophenol which can be monitored by a photometer at 405 nm. An E. coli XL-MRF' strain cell suspension without hpAP or ciAP-plasmid was used as a neg. control.

### Example 4

### Mutant and Chimeric Clones Obtained

The results of AP activity testing and corresponding clone analysis by sequencing are given in the following table:

| | | | Activity in | Remarks |
|---|---|---|---|---|
| Clone | Chimerization | Mutation | ΔA/min | |
| 1 | ciAP to hpAP at 478 | V69A | 0 | — |
| 2 | Whole protein as ciAP | — | 0.033 | — |
| 3 | ciAP to hpAP at 130 and | — | 0 | Primer SEQ ID NO: 7 did not |
| | from hpAP to ciAP at 165 | | | bind, 32 aas not sequenced |
| 4 | Whole protein as hpAP | Q41E | 0 | Primers SEQ ID NO: 7 and |
| | | | | SEQ ID NO: 8 did not bind, |
| | | | | 28 aas not sequenced |
| 5 | ciAP to hpAP at 508 | S136I, S507T | 0.006 | — |
| 6 | Whole protein as ciAP | D200N, Q202H | 0.006 | — |
| 7 | Whole protein as ciAP | Deletion from 497 to the end | 0.005 | — |
| | | (6 amino acids) | | |
| 8 | Whole protein as ciAP | T436A, G403S | 0.016 | — |
| 9 | Whole protein as ciAP | D218G | 0 | — |
| 10 | ciAP to hpAP at 478 | I375F | 0 | — |
| 11 | ciAP up to 150 then | | 0 | — |
| | random sequence | | | |
| 12 | ciAP to hpAP at 156 | K104R | 0.07 | Primers SEQ ID NO: 7 and |
| | | | | SEQ ID NO: 8 did not bind; |
| | | | | 28 aas not sequenced |
| 13 | Whole protein as ciAP | — | 0.013 | — |
| 14 | Whole protein as ciAP | K203, E438K, I497S | 0.004 | — |
| 15 | Whole protein as ciAP | G128D | 0 | — |
| 16 | ciAP to hpAP at 37 and | Insertion at 443 of 23 amino | 0 | — |
| | from hpAP to ciAP at 43 | acids (doubling of 23aa ciAP | | |
| | | sequence at 443) | | |
| 17 | Whole protein as ciAP | — | 0.007 | — |
| 18 | no data | no data | 0 | — |
| 19 | no data | no data | 0 | — |
| 20 | ciAP to hpAP at 478 | M356V | 0.015 | — |

It should be noted that hpAP is the starting template. Numbers refer to amino acid positions in the AP enzyme. Expressions like V69A mean an amino acid exchange at position 69 from V (valine) to A (alanine). The alignment of the sequences on an amino acid basis is shown in FIG. 2.

The above data show that chimerization occurred to almost any desired extent. The mutant spectrum ranges from no gene mixture at all (hpAP) to a complete reassembly of the fragmented ciAP gene.

Of the 20 clones isolated, 50% showed alkaline phosphatase activity, also including mutants with mixed DNA. In fact the clone with the highest activity (No. 12) is a chimaera. Point mutations, insertions and deletions were observed in addition to the chimerization further extending the pool of novel polynucleotides on which selection for novel or improved properties can be based.

1. A method for generating a recombinant polynucleotide, the method comprising the steps of:
(a) providing a closed circular polynucleotide template comprising a first gene of interest,
(b) providing fragments of a double-stranded DNA target polynucleotide derived from a second gene of interest, wherein the second gene of interest hybridizes to the first gene of interest, the fragments having free 3′-OH ends and phosphorylated 5′-ends,
(c) generating single strands of both the template polynucleotide and the fragments of the target polynucleotide,
(d) annealing the fragments of the target polynucleotide to the template polynucleotide,
(e) elongating the annealed fragments of the target polynucleotide,
(f) ligating the nicks between the elongated fragments wherein a recombinant circular DNA molecule is generated,
(g) generating single strands of both the recombinant circular DNA molecules obtained in (f) and the fragments of the target polynucleotide,
(h) annealing the fragments of the target polynucleotide to the recombinant circular DNA molecules obtained in (g),
(i) elongating the fragments of the target polynucleotide annealed in step (h), and
(j) ligating the nicks between the fragments elongated in step (i).

2. The method of claim 1 wherein steps (g) through (i) are performed 1 to 100 times to generate a mutated recombinant circular DNA molecule.

3. The method of claim 1 wherein steps (g) through (i) are performed 1 to 100 times to generate a chimaeric recombinant circular DNA molecule.

4. The method of claim 1 wherein steps (g) through (i) are performed 1 to 100 times to generate a mutated and chimeric recombinant circular DNA molecule.

5. The method of claim 1 wherein a thermostable DNA polymerase is used for elongating the annealed fragments of the target polynucleotide.

6. The method of claim 1 wherein a thermostable DNA ligase is used for ligating the nicks between the elongated fragments.

7. The method of claim 1 wherein the first gene of interest and the second gene of interest are variants of a single gene.

8. The method of claim 1 wherein two or more variants of the first gene of interest are provided.

9. The method of claim 1 wherein two or more variants of the second gene of interest are provided.

10. The method of claim 1 wherein the template polynucleotide is a double-stranded closed circular plasmid comprising an origin of replication, a promoter for gene expression, a gene for selection, and the first gene of interest.

11. The method of claim 1 wherein the first gene of interest comprises methylated DNA.

12. A method for obtaining a transformed host cell, the method comprising the step of introducing a recombinant polynucleotide obtained according to the method of claim 1 into the host cell.

13. The method of claim 12 wherein the recombinant polynucleotide is used for transformation without prior purification.

14. A method for obtaining a transformed host cell, the method comprising the step of introducing a recombinant polynucleotide obtained according to the method of claim 2 into the host cell.

15. The method of claim 14 wherein the recombinant polynucleotide is used for transformation without prior purification.

16. A method for obtaining a transformed host cell, the method comprising the step of introducing a recombinant polynucleotide obtained according to the method of claim 3 into the host cell.

17. The method of claim 16 wherein the recombinant polynucleotide is used for transformation without prior purification.

18. A method for obtaining a transformed host cell, the method comprising the step of introducing a recombinant polynucleotide obtained according to the method of claim 4 into the host cell.

19. The method of claim 18 wherein the recombinant polynucleotide is used for transformation without prior purification.

20. A host cell comprising a recombinant polynucleotide produced according the method of claim 1.

21. A host cell comprising a recombinant polynucleotide produced according the method of claim 2.

22. A host cell comprising a recombinant polynucleotide produced according the method of claim 3.

23. A host cell comprising a recombinant polynucleotide produced according the method of claim 4.

24. A method for obtaining a recombinant bio-molecule, the method comprising the steps of:
(a) obtaining a recombinant polynucleotide using the method of claim 1,
(b) transforming the recombinant polynucleotide into an appropriate host cell,
(c) expressing the recombined gene, and
(d) screening for the expression of the recombinant bio-molecule.

25. The method of claim 24 wherein the recombinant bio-molecule is an enzyme.

26. A method for obtaining a recombinant bio-molecule, the method comprising the steps of:
(a) obtaining a recombinant polynucleotide using the method of claim 2,
(b) transforming the recombinant polynucleotide into an appropriate host cell,
(c) expressing the recombined gene, and
(d) screening for the expression of the recombinant bio-molecule.

27. The method of claim 26 wherein the recombinant bio-molecule is an enzyme.

28. A method for obtaining a recombinant bio-molecule, the method comprising the steps of:
(a) obtaining a recombinant polynucleotide using the method of claim 3,
(b) transforming the recombinant polynucleotide into an appropriate host cell,
(c) expressing the recombined gene, and
(d) screening for the expression of the recombinant bio-molecule.

29. The method of claim 28 wherein the recombinant bio-molecule is an enzyme.

30. A method for obtaining a recombinant bio-molecule, the method comprising the steps of:
(a) obtaining a recombinant polynucleotide using the method of claim 4,
(b) transforming the recombinant polynucleotide into an appropriate host cell,
(c) expressing the recombined gene, and
(d) screening for the expression of the recombinant bio-molecule.

31. The method of claim 30 wherein the recombinant bio-molecule is an enzyme.
